(19) United States
(12) Patent Application Publication
(10) Pub. No.: US 2019/0183620 A1
(43) Pub. Date: Jun. 20, 2019

(54) CURING LIGHT WITH INTEGRATED FEEDBACK SENSOR

(71) Applicant: Garrison Dental Solutions, LLC, Spring Lake, MI (US)

(72) Inventor: Steven H. Peterson, Martin, MI (US)

(21) Appl. No.: 16/218,619

(22) Filed: Dec. 13, 2018

Related U.S. Application Data
(60) Provisional application No. 62/598,832, filed on Dec. 14, 2017.

(51) Int. Cl. A61C 13/15; F21V 23/00

(52) U.S. Cl. CPC A61C 19/004; A61N 2005/0652; F21V 23/003

(57) ABSTRACT

A light curing instrument for curing a target (with a material that is curable in response to application of light energy) includes a light source capable of outputting the light energy along an illumination path to cure the material and a sensor. The light source is controllable to vary the light energy being output, and the sensor, such as a photodiode, is configured to sense a light characteristic of light reflected back from the target. The sensor senses light reflected back along a sensing path and generates a feedback sensor signal. The instrument further includes a substrate for supporting the light source and the sensor, which is configured to support the light source and the sensor so that the illumination path and the sensing path are co-axial.

This application claims the benefit of U.S. Prov. App. Ser. No. 62/598,832, entitled CURING LIGHT WITH INTEGRATED FEEDBACK SENSOR, filed Dec. 14, 2017, which is incorporated by reference herein in its entirety.

### BACKGROUND

For dental curing lights utilizing blue LEDs as the illumination source, the low optical power capabilities and the high cost of the new blue LED technology were a limiting factor for several years. However, it was not so long until LED curing wands providing a reliable 200-300 mw/cm² (milliwatts per sq. centimeter) were technically viable and commercially available. This allowed the introduction of small hand-held battery operated curing lights that could perform 20-60 second composite cure times like their older cord based halogen predecessors.

As it is with most products and processes, there is a constant pressure for better, faster, stronger solutions. The LED curing light is no exception. The present state of the art now has hand-held battery-operated curing lights capable of producing more than 10 times that power—several at 3,200 mw/cm² or more, and some now as high as 8,000. This results in a curing light that can, in theory, cure a composite placement in a mere 3 to 5 seconds by delivering the required total optical energy (usually measured in joules) in less than one tenth the time of earlier generation curing lights. So then, with the introduction and subsequent market penetration of high powered LED curing lights to be used for class 2 and other forms of restorative dentistry, very significant gains were made in the ability to cure the composite fillings in mere seconds instead of 2-3 minutes of optical curing time.

In the past, prior to availability of the higher power curing lights (>1200 mW/cm²), dentists would typically compensate for the significant variances in the total optical energy delivered to the intended restoration (usually caused by positional and angular variations of the curing light relative to the intended target) by simply curing with second, third, and often times even additional doses of the full recommended curing exposure. This multiple exposure practice quite effectively avoided the potential drawbacks of an under-cured restoration at the cost of merely spending a couple extra minutes on the light cure process. However, with the advent of newer high-power curing lights over the past several years a new concern has developed of over exposure. Over exposure with the higher curing light power levels could now potentially overheat the tooth and cause permanent damage to healthy dental pulp tissue—especially when the old practice of extra curing is utilized as the primary means to avoid under exposure as was done in the past.

As disclosed in copending U.S. patent application Ser. No. 14/857,273, filed on Sep. 17, 2015, entitled DENTAL CURING LIGHT and Ser. No. 15/797,801, filed on Oct. 30, 2017, entitled DENTAL CURING LIGHT, which are incorporated herein by reference in their entireties, closed loop control on the power generated by the LED source based on the actual irradiance hitting the tooth can eliminate the positional and angular variations that were the root cause of varied and imprecise curing exposure levels in the first place.

As described, the closed loop control solves this combined concern of under-cure and over-cure by sensing the actual irradiance at the targeted restoration, and then adjusting the power delivered by the LED in real time (i.e. hundreds of times per second) so as to effectively close the loop and deliver a known, consistent, and accurate level of irradiance and total energy (Joules) to the tooth independent of positional variations of the curing light relative to the tooth caused by the operator. While this closed loop curing light concept offers significant advancement in accurately controlling the dosimetry of dental curing at the intended target, there are challenges. The optical feedback system is not trivial or inexpensive to manufacture. One of the challenges is to provide adequate feedback signal levels indicative of irradiance at the targeted surface while at the same time eliminating undue optical signal “noise” via the undesired light directly or indirectly coming from the LED source that is not indicative of target irradiance, and it must do so with a high degree of spatial selectivity so as to sense the intended target's irradiance that needs to be controlled with minimal or no noise—in other words ideally to only sense the intended target's irradiance that needs to be controlled. Further, it must also do so without getting in the way (i.e. shadowing) or otherwise adversely impacting the uniform distribution of the curing beam put forth by the curing light's LED source.

Accordingly, there is a need to provide a curing light that can cure quickly while avoiding or minimizing under exposure or over exposure.

### SUMMARY

The present disclosure describes an optical feedback system with an integrated sensor, such as a photo diode, that is used as the feedback sensor and integrated onto the same chip substrate as the LED source die.

In one embodiment, a light curing instrument for curing a target (with a material that is curable in response to application of light energy) includes a light source capable of outputting the light energy along an illumination path to cure the material and a sensor. The light source is controllable to vary the light energy being output, and the sensor, such as a photodiode, is configured to sense a light characteristic of light reflected back from the target. The sensor senses light reflected back along a sensing path and generates a feedback sensor signal. The instrument further includes a substrate for supporting the light source and the sensor, which is configured to support the light source and the sensor so that the illumination path and the sensing path are co-axial.

In one aspect, the light source comprises a plurality of light emitting diodes, optionally four light emitting diodes, with the light emitting diodes arranged in an array, and the sensor located between the light emitting diodes.

In another aspect, the sensor is centrally located between the light emitting diodes.

In yet another embodiment, the light curing instrument further includes a lens extended over the light source and the sensor, with the lens optionally including an anti-reflective coating.

In a further embodiment, the light curing instrument further includes an optically transmissive element optically coupled to the sensor and the lens.

For example, the side of the lens facing the light source and the sensor may include a recess, with the transmissive element extending into the recess on one terminal end thereof and optically coupled to the sensor in another terminal end thereof.

In one embodiment, the sensor is optically coupled to the optically transmissive element at an interface, and the light curing instrument further includes a shield, such as an opaque sealant, over the interface.

According to yet other aspects, the light curing instrument further includes a controller operably coupled to the light sensor and the light source. The controller is configured to vary, based on the light characteristic sensed by the light sensor, an operating characteristic of the light source to affect the light energy being output from the light source.

Optionally, the controller is configured to use the feedback sensor signal of the sensor to provide a time integral of the optical intensity on the target and compute, in real time, the actual total energy delivered to the target's surface.

In any of the above, the sensor may be configured, such as by positioning or shielding, to substantially avoid shadowing the illumination path.

### DETAILED DESCRIPTION OF THE DRAWINGS

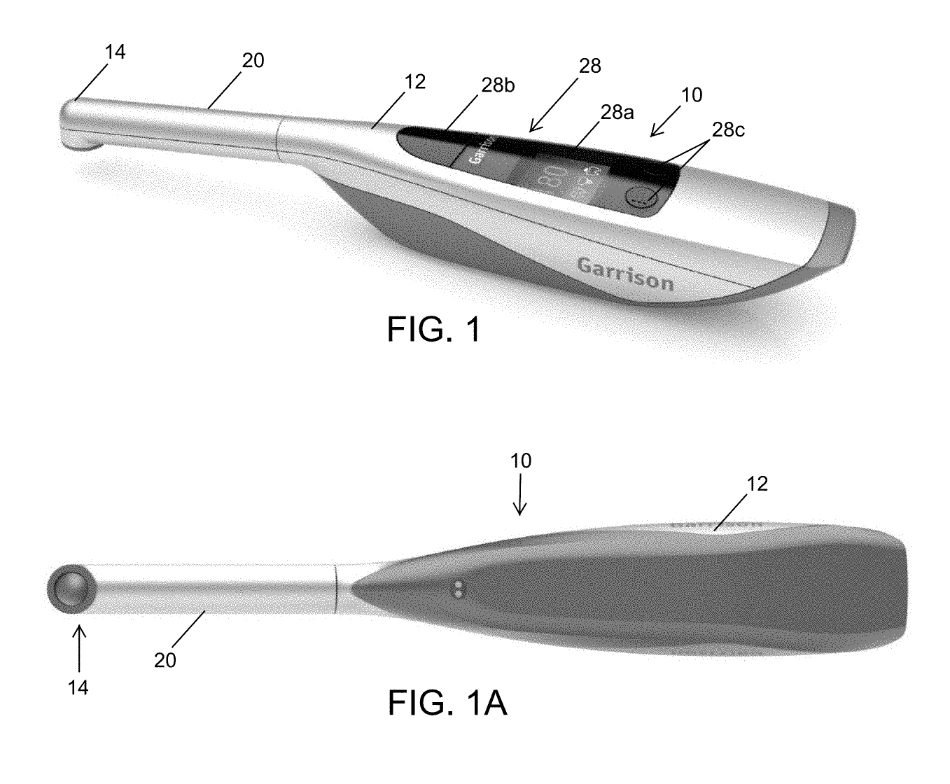

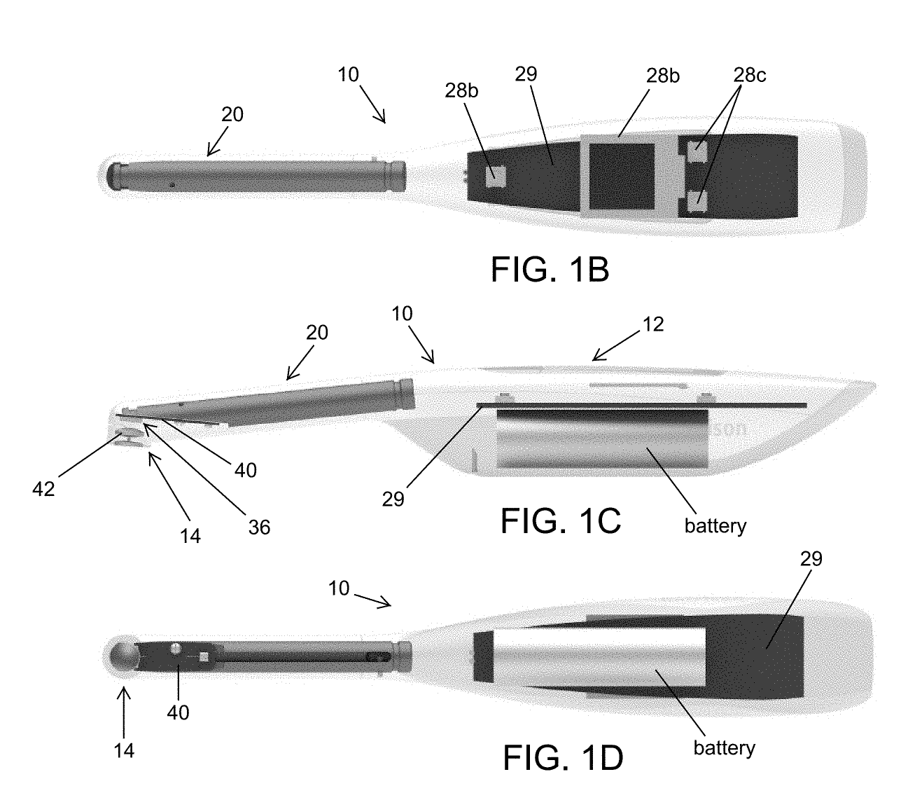

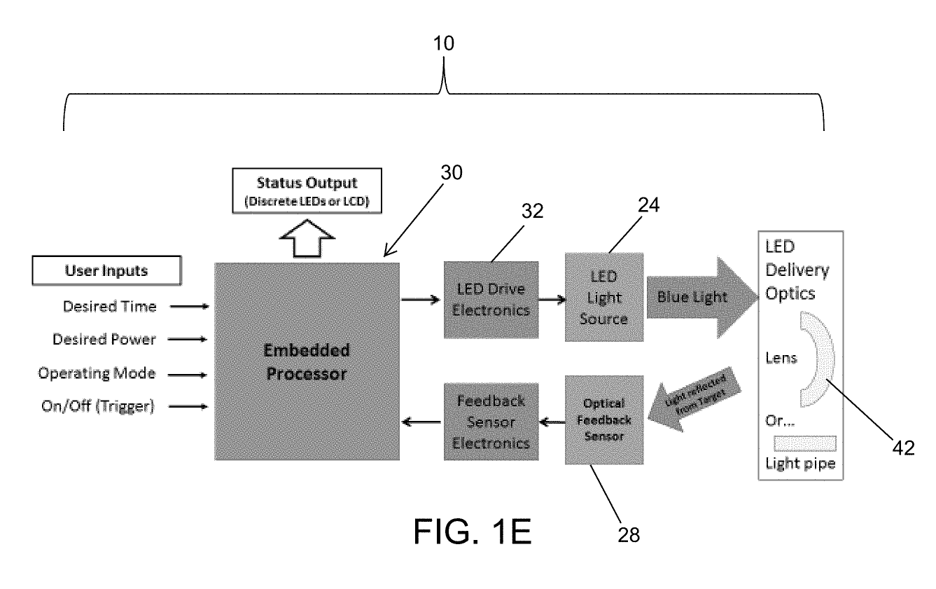

FIG. 1 is a perspective view of a light curing instrument according to one embodiment;

FIG. 1 A is a bottom plan view of the light curing instrument of FIG. 1;

FIG. 1B is a fragmented, partially sectioned view as viewed from the top of the light curing instrument of FIG. 1;

FIG. 1C is a fragmented, partially sectioned view as viewed from the side of the light curing instrument of FIG. 1;

FIG. 1D is a fragmented, partially sectioned view as viewed from the bottom of the light curing instrument of FIG. 1;

FIG. 1E is a schematic drawing of the light curing instrument of FIG. 1 illustrating the control system;

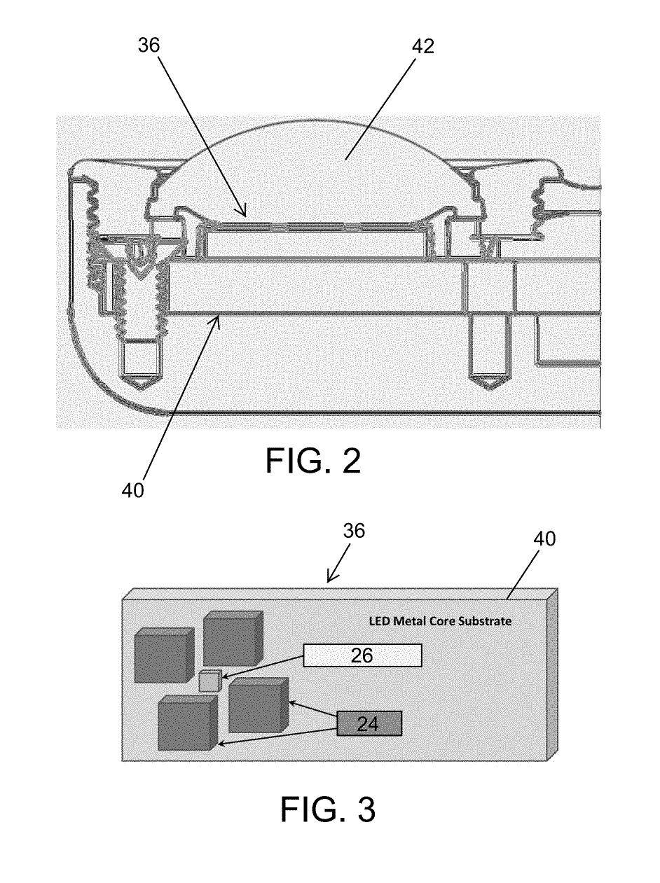

FIG. 2 is an enlarged section view of the light application end the light curing instrument of FIG. 1;

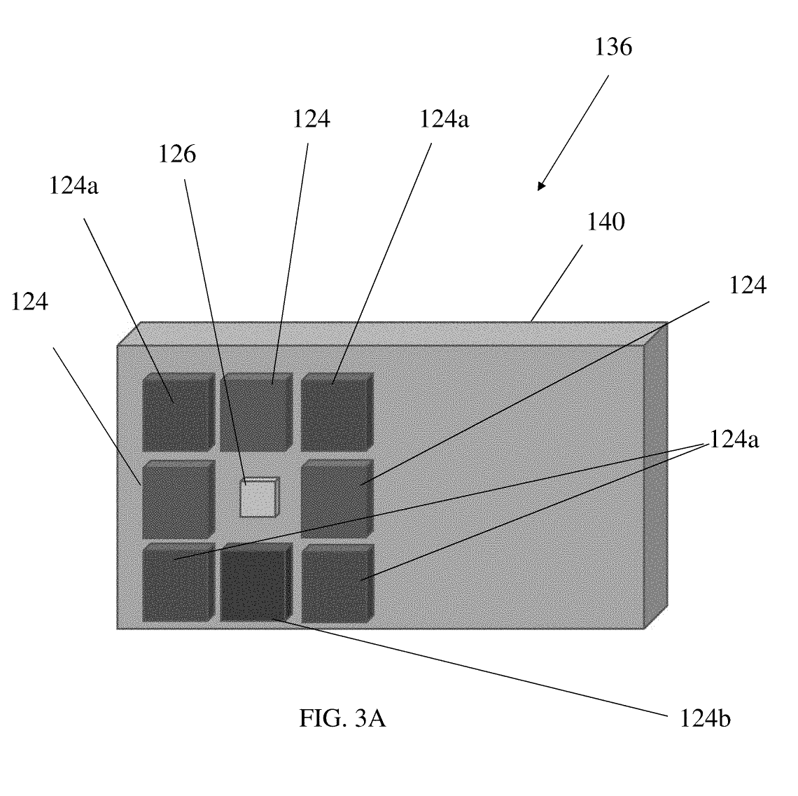

FIG. 3 is an enlarged view of one embodiment of the sensor and light source layout of the light application and sensing assembly of the light curing instrument;

FIG. 3A is an enlarged view of another embodiment of the sensor and light source layout of the light application and sensing assembly of the light curing instrument;

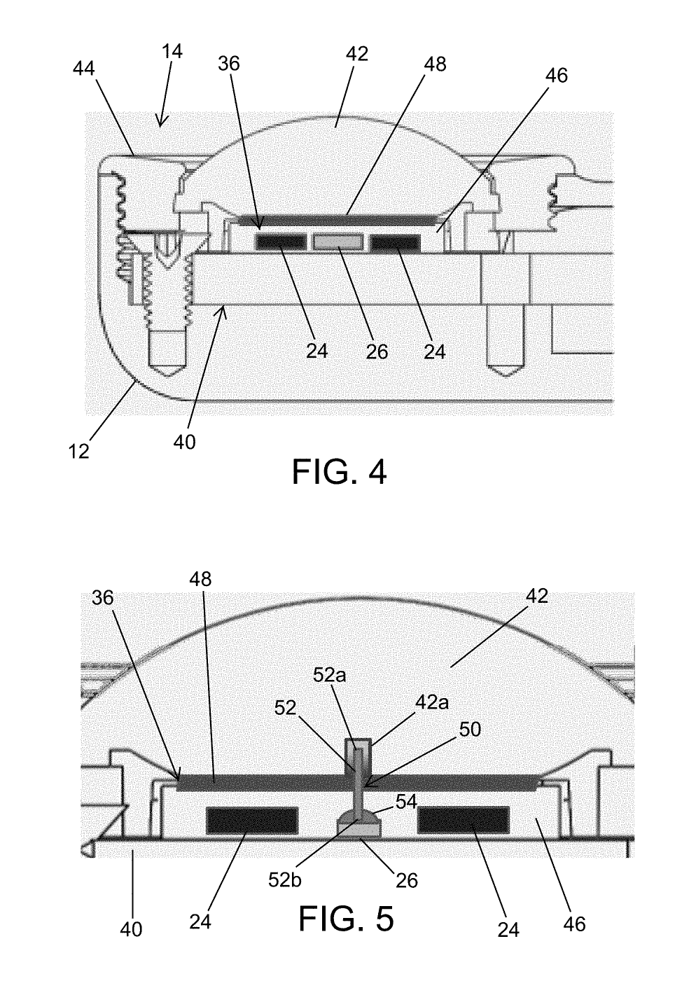

FIG. 4 is an enlarged section view of another or second embodiment of the light application end the light curing instrument;

FIG. 5 is an enlarged section view of third embodiment of the light application end the light curing instrument; and

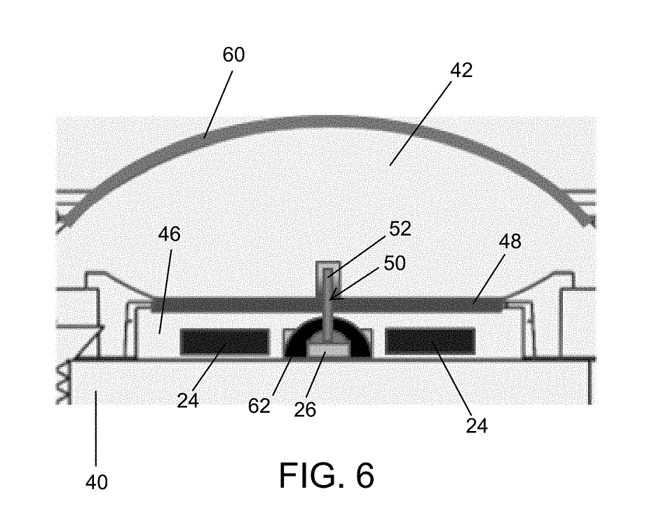

FIG. 6 is an enlarged section view of fourth embodiment of the light application end the light curing instrument.

### DETAILED DESCRIPTION OF EMBODIMENTS

Referring to FIG. 1, the numeral 10 generally depicts a light curing instrument for providing light to a composite material during a cure. Curing instrument 10 may be used to cure a light activated composite material, such as by polymerizing monomers into durable polymers. Curing instrument 10 may be a standalone device, such as a portable handheld wand having a battery power source and controls, or a component of a curing system having a base unit to which the curing instrument 10 is tethered and receives power therefrom and optionally control signals therefrom. A variety of fields may benefit from the curing instrument 10, including, for example, the dental and medical fields and non-dental fields, such as industrial manufacturing where precise light cure of adhesives or similar composite fills are required or at least desired. For purposes of disclosure, curing instrument 10 is described as being a dental curing instrument for use in connection with curing a composite material having photo initiator, which absorbs light of a particular wavelength and causes polymerization of the monomers included in the composite material into polymers. It should be understood, however, that the present disclosure is not limited to the curing instrument being a dental curing instrument or limited to use with dental composite material—any curing application may benefit from the curing instrument, and any type of photo curable material may be used in conjunction with the curing instrument, including transparent, translucent and semi-opaque curable materials.

Referring again to FIGS. 1 and 1A-1D, curing instrument 10 includes a housing 12, which houses the various electronics and electrical components described below and which includes a narrowed, elongate neck (light application member 20) whose end forms a light application end 14 that supports light sources 24 and a feedback sensor 26 (e.g. FIG. 3), such as a photodiode, both more fully described below. Further, housing 12 optionally supports an operator interface 28 with a display 28a and an operator input device 28b, such as a button, which allows an operator to turn the curing instrument on or off and optionally additional operator input devices 28c, which may all be commonly mounted on a printed circuit board PCB 29 (FIGS. 1B-1D) to allow the operator selected between operating modes or adjust one or more operating parameters of the curing light. Light curing instrument 10 may be a standalone unit or be coupled to a control unit or the like, which control unit may instead include the operator interface and/or operator input device. In use, an operator may activate the curing instrument 10 via the operator interface 28 (e.g. using a start button) to initiate a curing operation of a composite material. After activation, the curing instrument 10 generates and emits light through a light passage of the application member 20 at the light application end 14 of housing 12. The operator may position the light application member 20 such that the light passage directs light toward the composite material in order to effect a cure thereof.

To control operation of light sources 24, curing instrument 10 includes a controller 30 (e.g., an embedded controller, such as an embedded microprocessor-based controller), which is in communication with sensor 26 and includes a drive circuitry 32 for driving light sources 24. The drive circuitry 32 controls the supply of power to the light sources 24 to generate light that is transmitted via the light application member 20 to the target surface. For instance, the drive circuitry 32 may include control drive circuitry that receives power from a power source (e.g., a battery of the curing instrument 10 or a hard wired power supply line), and provides that power as a power signal to the light source 24 according to one or more operating characteristics, such as a voltage magnitude, current magnitude, or duty cycle or a combination thereof. In response to receipt of power, the light sources 24 generate light that can be directed to the target or targeted surface for the curing operation. The light sources 24, in the illustrated embodiment, are primarily a deep blue and/or an Ultra-Violet (UV) light source, such as a UV light emitting diode (an LED that produces the shorter wave lengths of blue light), but may be configured differently. It should further be understood that the light sources 24 in the illustrated embodiment—although primarily one type of light source (e.g., UV)—also may emit light of wavelengths different from those of the primary light type. For instance, the primary light output from a UV LED is UV light, but the UV LED may also emit light in the visible spectrum or infrared spectrum, or both, along with the UV light.

The controller 30 of the curing instrument 10 in one embodiment may include an algorithmic computational solution element or controller module, such as a shared computational module incorporated into the controller 30, forming an embedded control system that controls light output and potentially additional instrument functionality. Optionally, this module may be separate from the controller 30 and incorporated into another hardware module that along with the controller 30 forms at least part of a control system for the curing instrument 10.

Control over generation of light from the light sources 24, as mentioned above, is conducted through the drive circuitry 32, which is also referred to as an LED power control element but is not so limited. In the illustrated embodiment, the controller 30 may be coupled to and control operation of the drive circuitry 32. The controlled level of the operating characteristic or operating characteristics of the drive circuitry 32 is governed at least in part by the controller 30 to control the power signal provided to the light sources and to control the light output thereof. For example, the controller 30 may provide a control signal or control information to the drive circuitry 32 to provide power to the light sources 24 according to a target operating characteristic. As will be more fully described below, the control signal or control information provided from the controller 30 may be dynamic such that, during a curing operation, the control signal or control information may vary to effect a change in the target operating characteristic.

The drive circuitry 32, in one embodiment, may utilize feedback circuitry to achieve the target operating characteristic. For instance, the drive circuitry 32 may include a current sensor that senses current supplied to the light sources 24, and based on the sensed current, the drive circuitry 32 may adjust operation to vary the supply current to more closely align with a target supply current.

Additionally or alternatively, as noted above curing instrument 10 may include a feedback sensor 26 in communication with controller 30 so that controller 30 may direct operation of the drive circuitry 32 based on sensed information by sensor 26 by adjusting one or more target operating characteristics, such as duty cycle. For further details of the control system and optional feedback control, reference is made to U.S. patent application Ser. No. 14/857,273, filed on Sep. 17, 2015, entitled DENTAL CURING LIGHT and Ser. No. 15/797,801, filed on Oct. 30, 2017, entitled DENTAL CURING LIGHT.

Referring to FIGS. 2 and 3, and as noted above, light sources 24 and feedback sensor 26 are commonly mounted on a substrate 40, which is then covered by a lens 42, such as a beam shaping lens, which is mounted in the opening of the light application end 14 of housing 12 by a bezel 44 over the light application and sensing assembly 36 formed by the light sources and sensor (and their mounting structures described below). In the illustrated embodiment, and as shown in FIG. 3, curing instrument 10 includes four LEDs (for example, without their own respective lenses, sometimes referred to as LED dies—optionally though they may include their own lenses) which are arranged in array on substrate 40 (i.e., a printed circuit board PCB). It should be understood that the number of LEDs may vary—and may include, for example, 8 LEDs.

As one example, as described below in reference to FIG. 3A, inclusion of 8 or more LED die would be especially useful if multiple LED output wavelengths were desired for curing or other non-curing functionality. Referring to FIG. 3A, exemplifies one such possible layout wherein 4 LED die with a non-curing red output wavelength are interspersed with blue & UV curing die for the purpose of preheating the restorative composite prior to application of light from the blue/UV wavelengths used for curing.

Sensor 26 is also mounted to substrate 40 between the LEDs and optionally centrally located or co-located adjacent the LEDs, and further covered by a silicone layer 46. A central or adjacent co-location of the sensor amongst the LEDs causes minimal expansion to the locus of the LEDs due to relatively small size of the sensor compared to the LEDs. However, central location of the sensor 26 supplies the greatest degree of coaxial alignment between the LED illumination beam and the sensor's viewing cone of sensing.

To improve the signal strength, signal to noise ratio (SNR), and/or spatial selectivity, and now referring to FIG. 4, curing instrument 10 optionally includes an optical bond 48 between the silicone layer 46 (which contains the LEDs 24 and sensor 26) and the beam shaping lens 42 over the light application and sensing assembly. The optical bond helps to provide higher optical efficiency for the LEDs (i.e. higher irradiance at any given LED power level), and it also helps to improve signal strength and SNR at the sensor.

Referring again to FIG. 3A, the numeral 136 refers to a second embodiment of the light application and sensing assembly. Light application and sensing assembly 136 includes light sources 124, feedback sensor 126, and one or more red LEDs 124a (LEDs that output light in the red spectrum) also all optionally commonly mounted on a substrate 140. For further optional details of light sources 124 and sensor 126 reference is made to the above embodiment. In the illustrated embodiment, light application and sensing assembly 136 includes four red LEDs 124a, which emit light of wavelengths in the infrared spectrum. Optionally, one of the light sources 124b may have a shorter wavelength than the other light sources 124. For example, light sources 124 may comprise a medium wavelength blue LED, while light source may 124b may comprise a shorter wavelength blue or violet LED. For further details of a suitable lens (42), which may be mounted in the opening of the light application end 14 of housing 12 by a bezel (e.g. bezel 44) over the light application and sensing assembly 136 reference is made to the first embodiment. As noted above, LEDs 124a (with a non-curing red output wavelength) may be interspersed with blue & UV curing die for the purpose of preheating the restorative composite prior to application of light from the blue/UV wavelengths used for curing.

Referring to FIG. 5, curing instrument 10 optionally may provide an optically controlled light path between the targeted area of the beam of LEDs and the sensor. For example, in the illustrated embodiment, curing instrument 10 may include an optical element 50, such as an optical fiber, that can provide a means to manage the spatial selectivity of the area being “viewed” by the sensor. For example, a short optical fiber 52 (such as shown in FIG. 5) is placed between the sensor and a small blind hole or recess 42a located in the bottom of the lens 42. The proximal terminal end 52a of fiber 52 is extended into the recess 42a, while the distal terminal end 52b of the fiber 52 is optionally coupled to the sensor 26 by a clear optical bonding agent 54 on top of sensor to improve signal strength at the sensor. As such, the surface finish on the distal terminal end (i.e., bottom end) of the fiber may be polished or not. The proximal terminal end (i.e., the top end) of the fiber is optionally polished or has a very cleanly “cleaved” end. This, along with an optically smooth surface at the blind end of the hole or recess in the lens, creates an air gap that allows the numerical aperture (NA) of the fiber to then control the viewing angle of the viewing cone that naturally emanates from the end of the fiber toward the targeted area of the LED beam. In similar fashion, the diameter of the short fiber will allow management of the signal strength delivered to the sensor as the “collection area” of the sensing fiber goes up with the square of the diameter.

To manage the field of view that is sensed by the sensor, the diameter of the fiber may be adjusted. The Effective Numerical Aperture (NA) of the fiber, as determined by the characteristic index of refraction exhibited by the core of the fiber (typically a fused silica, but not limited to same) determines the angle of the sensed field of view as it proceeds outward from the fiber's polished end. The diameter of the fiber directly impacts the surface area from which optical feedback is collected, and therefore impacts the optical signal strength of the reflected feedback signal that is directed to the sensor. The signal strength variation is approximately proportional to the square of the fiber diameter. Also, the height of the shield fiber (described below) aids in reducing undesired optical noise by extending beyond the silicone layer that seals and protects the LED and sensor on the substrate. As such it avoids inclusion of internal rays reflected off the inner surface of said silicone layer. The height of the shielded fiber likewise aids in avoiding first surface or optical boundary interface retro-reflections from first surface of the lens located adjacent to the protective surface of the LED. The height of the shielded fiber also allows some “tuning” of where the viewing cone, whose inclusion angle is determined by the fiber NA, begins to take effect and spread out toward the feedback target plane. (i.e., some depth below the outer surface of the objective lens is desired to help minimize the percent of viewing spot size as a function of distance between tip and targeted tooth). For example, the height of the fiber may be typically at least 5 to 10 times the diameter of the fiber or at least 1 mm. Further, the fiber may extend all the way through the lens but then would be optionally covered with a glass cover to prevent intrusion and/or scratching. The diameter of the fiber may be at least or about 10 microns.

Referring to FIG. 6, in yet another embodiment, optionally curing instrument 10 includes an optical barrier 62 between the LEDs 24 and the sensor 26 and further optionally one that forms a barrier between the sensor's interface with the fiber 52 and LEDs 24 to provide a good SNR. The barrier 62 may be formed by an opaque layer of light cured or self-curing material over the top of the sensor and its interface with the fiber 52. In the illustrated embodiment, the opaque layer comprises a black layer than forms a dome over the sensor and sensor/fiber interface. With the arrangement, there is a great reduction of noise to the optical feedback signal (signal generated by sensor 26) by eliminating almost all of the light coming directly from the LEDs that would otherwise have been noise to the desired feedback signal. The control system can be further enhanced in SNR and delivered irradiance by also coating the outside surface of the lens 42 with an anti-reflective (AR) coating 60, such as a UV-Visible light AR coating 60 or one that has been specifically tuned to the 405-460 nm range of blue curing lights. As a by-product, it is has been found that the AR coating also adds about 6-8% more actual light delivered on target at any given LED power level.

Accordingly, the curing light described herein may have one or more of several elements that can improve the signal strength, signal to noise ratio (SNR), and/or spatial selectivity of the curing instrument. As noted above, these elements may include: (1) Locating the sensor in the center (or close to the center) of the LED array to achieve a desired coaxial alignment of the feedback sensing cone with the intense curing beam projected from the curing instrument light application end; (2) an optically transmissive element to select and direct light from the surface of the intended target surface to the feedback sensor, which can also help manage the signal strength, SNR, and/or spatial selectivity of its light gathering function; (3) one or more optical bondings, for example, between to optically couple the optical transmissive element and feedback sensor with high transmissive efficiency; (4) one or more shielding coatings, which shield the sensor/fiber interface and/or the feedback sensor from most if not all light emitted from the LEDs; (5) one or more AR coatings over the lens 42, such as an AR coating that is tuned for the generic UV-Visible spectrum or specifically tuned for the 405-460 nanometer spectrum, which is highly effective in reducing undesired internal reflections which significantly improves the SNR; (6) a polished tip on the fiber element, which can add significant improvements to the spatial selectivity of the fiber element with respect to its directional sensitivity; and/(7) an optical bond between the light application and sensing assembly 36 (and its silicon protective coating) and the lens to help shape/concentrate the beam of the curing instrument, which is very effective at improving the efficiency of the illumination beam (as much as 30%), and it also contributes to a better SNR on the sensing of the feedback signal generated by the surface irradiance of the target at which the light is aimed.

We claim:

1. A light curing instrument for curing a target, the target including a material that is curable in response to application of light energy, the light curing instrument comprising:
a light source capable of outputting the light energy along an illumination path to cure the material, the light source being controllable to vary the light energy being output;
a sensor, such as a photodiode, to sense a light characteristic of light reflected back from the target, the sensor sensing light reflected back along a sensing path and generating a feedback sensor signal; and
a substrate for supporting the light source and the sensor, the substrate configured to support the light source and the sensor so that the illumination path and the sensing path are co-axial.

2. The light curing instrument according to claim 1, wherein the light source comprises a plurality of light emitting diodes, optionally four light emitting diodes, the light emitting diodes arranged in an array, and the sensor located between the light emitting diodes.

3. The light curing instrument according to claim 2, wherein the sensor is centrally located between the light emitting diodes.

4. The light curing instrument according to claim 1, further comprising a lens extended over the light source and the sensor, the lens optionally including an anti-reflective coating.

5. The light curing instrument according to claim 4, further comprising an optically transmissive element optically coupled to the sensor and the lens.

6. The light curing instrument according to claim 5, wherein the lens includes a side facing the light source and the sensor, the side of the lens including a recess, the transmissive element extending into the recess on one terminal end thereof and optically coupled to the sensor in another terminal end thereof.

7. The light curing instrument according to claim 5, wherein the sensor is optically coupled to the optically transmissive element at an interface, the light curing instrument further comprising a shield, such as an opaque sealant, over the interface.

8. The light curing instrument according to claim 1, further comprising a controller operably coupled to the light sensor and the light source, the controller configured to vary, based on the light characteristic sensed by the light sensor, an operating characteristic of the light source to affect the light energy being output from the light source.

9. The light curing instrument according to claim 8, wherein the controller is configured to use the feedback sensor signal of the sensor to provide a time integral of the optical intensity on the target and compute, in real time, the actual total energy delivered to the target's surface.

10. The light curing instrument according to claim 1, wherein the sensor is configured to substantially avoid shadowing the illumination path.

11. A light curing instrument for curing a target, the target including a material that is curable in response to application of light energy, the light curing instrument comprising:
a first light source capable of outputting light energy in the blue or violet spectrum to cure the material, the light source being controllable to vary the light energy being output;
a sensor, such as a photodiode, to sense a light characteristic of light reflected back from the target, the sensor sensing light reflected back from the material and generating a feedback sensor signal; and
a second light source configured for outputting light energy in the infrared spectrum to warm the material.

12. The light curing instrument according to claim 11, further comprising a substrate for supporting the first light source and the sensor, the substrate configured to support the light source and the sensor so that the illumination path and the sensing path are co-axial.

13. The light curing instrument according to claim 12, wherein the first light source comprises a plurality of light emitting diodes arranged in an array on the substrate.

14. The light curing instrument according to claim 13, wherein the sensor is centrally located between the light emitting diodes.

15. The light curing instrument according to claim 13, wherein at least one of the light emitting diodes outputs light at a lower frequency than the remaining light emitting diodes.

16. The light curing instrument according to claim 12, wherein the substrate supports the second light source.

17. The light curing instrument according to claim 11, further comprising a controller operably coupled to the first light source and the first light source, the controller configured to vary, based on the light characteristic sensed by the light sensor, an operating characteristic of the light source to affect the light energy being output from the light source.

18. The light curing instrument according to claim 17, wherein the controller is configured to use the feedback sensor signal of the sensor to provide a time integral of the optical intensity on the target and compute, in real time, the actual total energy delivered to the target's surface.

19. The light curing instrument according to claim 11 wherein the sensor is configured to substantially avoid shadowing the illumination path.

20. The light curing instrument according to claim 19 wherein the sensor is shielded to substantially avoid shadowing the illumination path.
